(19) United States
(12) Patent Application Publication
(10) Pub. No.: US 2016/0151067 A1
(43) Pub. Date: Jun. 2, 2016

(54) Vascular Clamp for Use in Minimally Invasive Surgery

(71) Applicant: METAL INDUSTRIES RESEARCH & DEVELOPMENT CENTRE, Kaohsiung City (TW)

(72) Inventors: Ming-Hui Chen, Kaohsiung City (TW); Ho-Chung Fu, Kaohsiung City (TW)

(21) Appl. No.: 14/557,527

(22) Filed: Dec. 2, 2014

(51) Int. Cl. A61B 17/122

(52) U.S. Cl. CPC A61B 17/122

(57) ABSTRACT

A vascular clamp for use in minimally invasive surgery includes a positioning member having a first side and a second side opposite to the first side and looping/tightening member and a reinforcing member. The positioning member includes a through-hole, a positioning hole, and a connection gap intercommunicating the through-hole with the positioning hole. Each of the through-hole, the positioning hole, and the connection gap extends from the first side through the second side of the positioning member. The looping/tightening member includes a connection end and a free end. The connection end is connected to the positioning member. The looping/tightening member includes a positioning section having a plurality of engagement blocks. A neck is interconnected between two adjacent engagement blocks. One of the engagement blocks is selectively and removably engaged in the positioning hole. The reinforcing member is mounted between the first and second sides of the positioning member.

### BACKGROUND OF THE INVENTION

1. Field of the Invention

The present invention relates to a vascular clamp and, more particularly, to a vascular clamp for use in minimally invasive surgery.

2. Description of the Related Art

Minimal trauma is opened in a body surface of a patient in minimally invasive surgery opens, and an endoscope is placed into the body of the patient during the surgical procedure. In comparison with ordinary surgical operations, minimally invasive surgery significantly reduces the trauma caused to the human body during the surgical procedure, eases the postoperative pain of the patient, and shortens the postoperative recovery period of the patient, such that the patient can get back to the normal life sooner. However, the surgical operator has a small field of vision during the surgery, and the surgical device has a small operating area, leading to an increase in the difficulties in the use of the surgical device.

During the minimally invasive surgery, the surgical operator generally uses a vascular clamp or a hemoclip that is pressed onto the peripheral wall of a blood vessel, such that the blood cannot flow in the blood vessel temporarily to permit proceeding of the surgery.

FIG. 1 shows a conventional vascular clamp 9 that can be pressed onto a peripheral wall of a blood vessel “V.” The conventional vascular clamp 9 is a C-shaped ring and includes two clamping jaws 91 and 92 on two ends thereof. An inner periphery of the conventional vascular clamp 9 defines a cavity 93 for receiving the blood vessel “V.” An example of such a conventional vascular clamp 9 is disclosed in U.S. Pat. No. 7,678,124.

The surgical operator can select a vascular clamp 9 of a suitable size according to the diameter of the blood vessel “V.” The blood vessel “V” is placed in the cavity 93 of the conventional vascular clamp 9 which then clamps the blood vessel “V” to temporarily block the flow of blood in the blood vessel V.

FIG. 2 shows a conventional hemoclip 8 pressed onto the peripheral wall of a blood vessel “V.” The conventional hemoclip 8 includes a V-shaped base portion 81 and two clamping portions 82 and 83 substantially parallel to each other. An opening 84 is defined by the base portion 81 and the two clamping portions 82 and 83 for receiving the blood vessel “V.” An example of such a conventional hemoclip 8 is disclosed in U.S. Pat. No. 4,646,740.

The surgical operator can place the conventional hemoclip 8 around an appropriate portion of the blood vessel “V” and then adjust the spacing between the two clamping portions 82 and 83 according to the diameter of the blood vessel “V.” Thus, the conventional hemoclip 8 can tightly clamp blood vessels “V” of various diameters to temporarily block the flow of blood in the blood vessels “V.”

However, a special surgical tool is required to remove the conventional vascular clamp 9 or the conventional hemoclip 8. In a long-term surgical procedure, death of cells surrounding the blood vessel “V” could occur if the blood cannot flow again in the blood vessel “V” in time. Thus, the surgical operator must spend extra time to remove the conventional vascular clamp 9 or the conventional hemoclip 8 during the surgical procedure, which not only extends the surgical procedure but results in difficulties in a surgical procedure on a patient with problems of obesity or intestinal adhesions. Furthermore, the postoperative recovery period could be prolonged.

FIG. 3 shows a conventional inflation type vascular clamp 7 including a tube 71. A loop 72 and a syringe 73 are connected to two ends of the tube 71. A gas is injected by the syringe 73 and flows through the tube 71 into the loop 72 to inflate the loop 72. The conventional inflation type vascular clamp 7 further includes a positioning member 74 located between the loop 72 and the tube 71. The loop 72 can be folded backwards and positioned on the positioning member 74 to form a circled space 721 for receiving a blood vessel “V.” A valve 731 is mounted between the tube 71 and the syringe 73 and can be opened and closed. An example of such a conventional inflation type vascular clamp 7 is disclosed in U.S. Pat. No. 4,531,519.

The surgical operator can wind the loop 72 around the blood vessel “V” and can fold the loop 72 backwards to position the loop 72 on the positioning member 74, forming the circled space 721. Then, the valve 731 is opened to inflate the gas into the tube 71 by the syringe 73. The gas flows through the tube 71 into the loop 72. The inflated loop 72 reduces the circled space 721 to an appropriate size to clamp the blood vessel “V”, and the valve 731 is then closed. If it is desired to loosen the blood vessel “V”, the valve 731 is opened again to activate the syringe 73 to suck the gas back from the loop 72 and the tube 71.

Since the size of the circled space 721 depends on the inflow or outflow of the gas and since the syringe 73 is located outside the body of the patient, the diameter of the tube 71 is generally made smaller due to restriction by the size of the minimal trauma of the minimally invasive surgery, such that the inflow speed and the outflow speed of the gas are limited by the reduced diameter of the tube 71, which could extend the surgical procedure.

Thus, improvement to the conventional vascular clamp 9, the conventional hemoclip 8, and the conventional inflation type vascular clamp 7 is required.

### SUMMARY OF THE INVENTION

An objective of the present invention is to provide a vascular clamp for use in minimally invasive surgery. The vascular clamp is not only suitable for blood vessels of various diameters but shortens the time for clamping and loosening the blood vessel, improving the efficiency of the minimally invasive surgery.

The present invention fulfills the above objective by providing a vascular clamp for use in minimally invasive surgery. The vascular clamp includes a positioning member having a first side and a second side opposite to the first side, a looping/tightening member and a reinforcing member. The positioning member includes a through-hole, a positioning hole, and a connection gap intercommunicating the through-hole with the positioning hole. Each of the through-hole, the positioning hole, and the connection gap extends from the first side through the second side of the positioning member. The looping/tightening member includes a connection end and a free end. The connection end is connected to the positioning member. The looping/tightening member includes a positioning section having a plurality of engagement blocks. A neck is interconnected between two adjacent engagement blocks. One of the plurality of engagement blocks is selectively and removably engaged in the positioning hole. The reinforcing member is mounted between the first and second sides of the positioning member.

Each of the through-hole, the positioning hole and the connection gap can extend through the reinforcing member.

The maximum outer diameter of each of the plurality of engagement blocks can be smaller than the minimum diameter of the through-hole and can be larger than the maximum diameter of the positioning hole. The maximum diameter of the connection gap can be smaller than the maximum diameter of the positioning hole.

The positioning member further includes a first end and a second end opposite to the first end. The first end of the positioning member can be connected to the looping/tightening member. The positioning hole can be located between the through-hole and the first end of the positioning member.

The positioning member can further include a gripping portion located between the through-hole and the second end of the positioning member.

The looping/tightening member can further include a looping section between the connection end and the positioning section. The looping section, a portion of the positioning section, and the positioning member together define a circled space. The circled space is adapted for receiving a blood vessel.

Each of the plurality of engagement blocks can have reducing outer diameters from the connection end towards the free end.

The looping/tightening member of the vascular clamp for use in minimally invasive surgery according to the present invention includes the engagement blocks that can selectively be engaged in the positioning hole to form a circled space suitable for blood vessels of various diameters, reducing the time required by the surgical operator for picking suitable devices according to the diameter of the blood vessel during the surgical procedure. Moreover, the time required by the surgical operator for loosening the blood vessel can be reduced since the minimum diameter of the through-hole is larger than the maximum outer diameter of each of the plurality of engagement blocks. Thus, the time of the surgical procedure can be reduced to improve the operational efficiency and to shorten the postoperative recovery period of the patient.

Furthermore, the reinforcing member mounted between the first and second sides of the positioning member can avoid deformation of the through-hole, the positioning hole, and the connection gap resulting from tearing, preventing disengagement of the engagement block positioned in the positioning hole.

The present invention will become clearer in light of the following detailed description of illustrative embodiments of this invention described in connection with the drawings.

### BRIEF DESCRIPTION OF THE DRAWINGS

The illustrative embodiments may best be described by reference to the accompanying drawings where:

FIG. 1 is a diagrammatic perspective view illustrating use of a conventional vascular clamp.

FIG. 2 is a diagrammatic perspective view illustrating use of a conventional hemoclip.

FIG. 3 is a diagrammatic perspective view of a conventional inflation type vascular clamp.

FIG. 4 is a perspective view of a vascular clamp for use in minimally invasive surgery of an embodiment according to the present invention.

FIG. 5 is a top view of the vascular clamp of FIG. 4.

FIG. 6 is a cross sectional view taken along section line A-A of FIG. 5.

FIG. 7 is a cross sectional view illustrating use of the vascular clamp according to the present invention with a looping/tightening member extended through a through-hole of a positioning member of the vascular clamp.

FIG. 8 is a cross sectional view similar to FIG. 7 with one of engagement blocks engaged in a positioning hole of the positioning member.

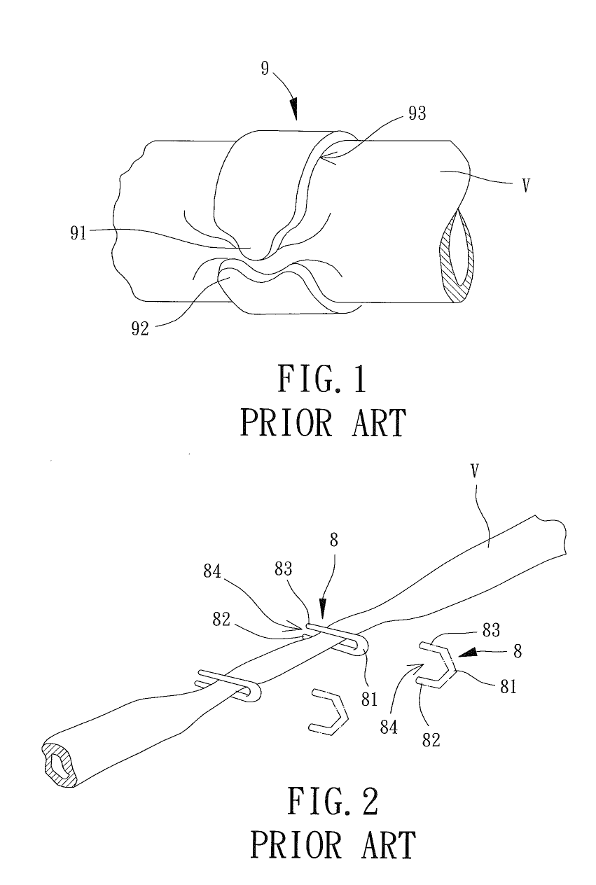

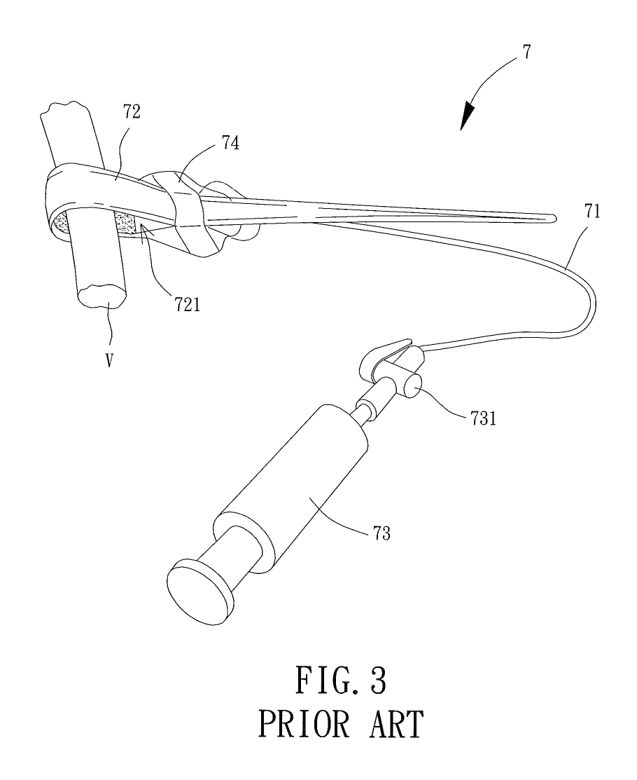

### DETAILED DESCRIPTION OF THE INVENTION

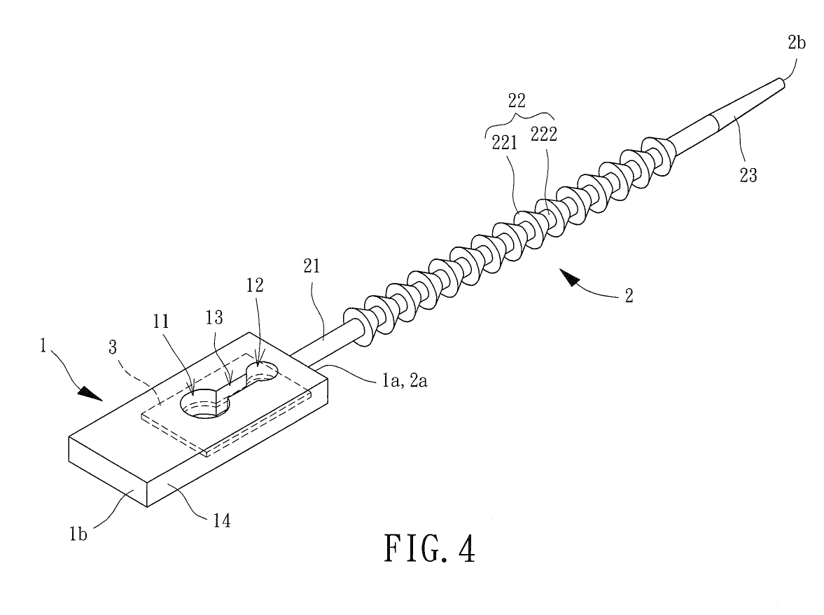

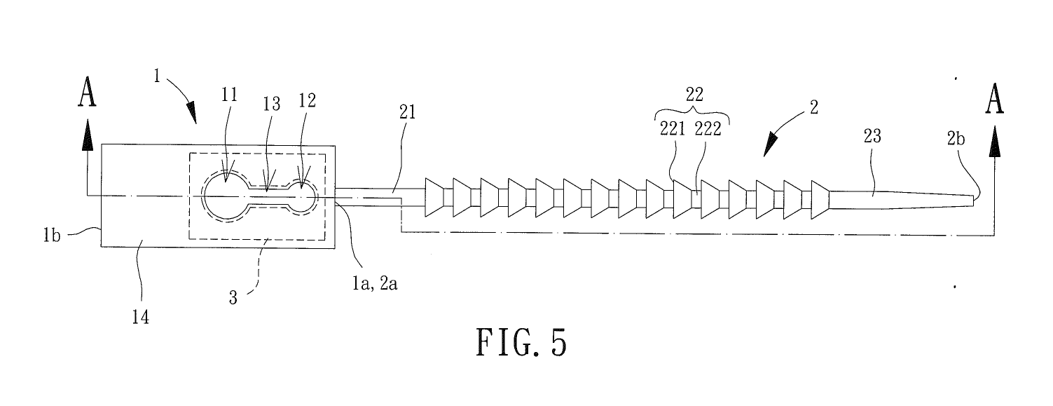

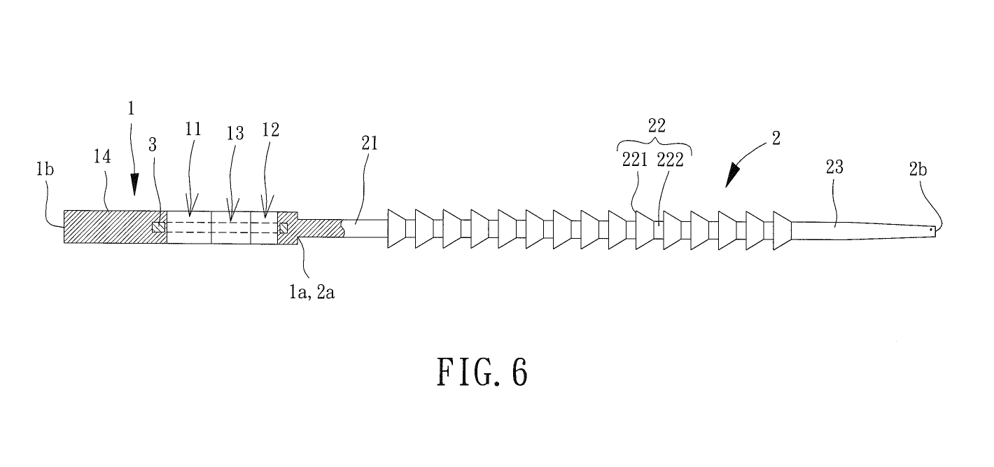

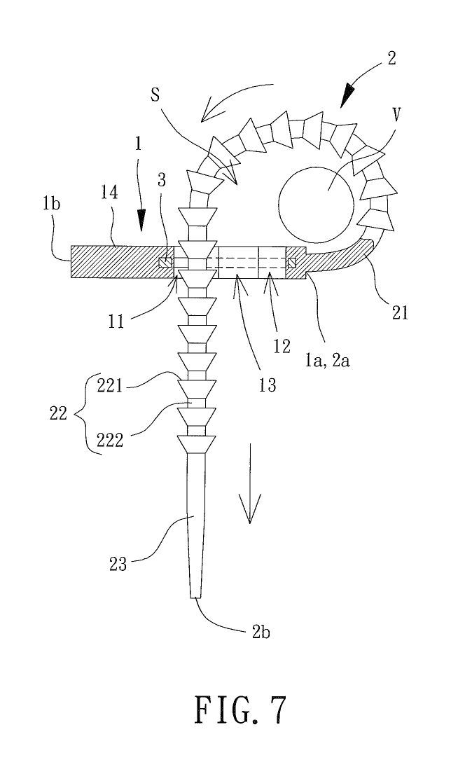

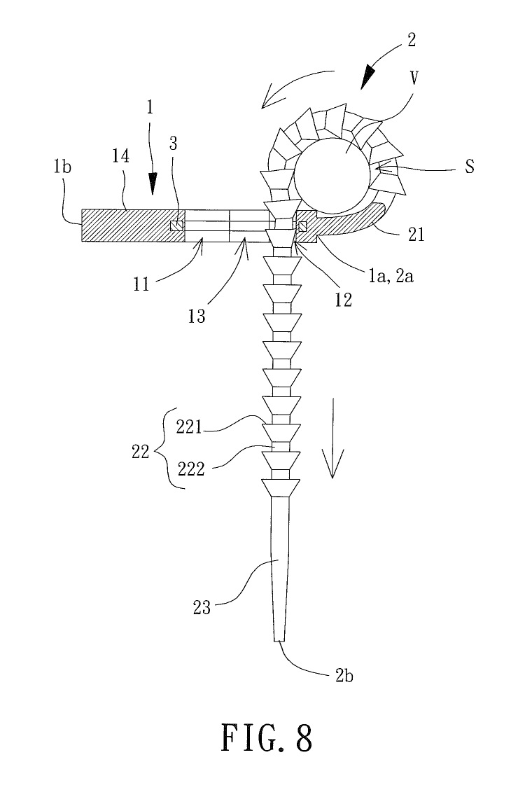

With reference to FIGS. 4-6, a vascular clamp for use in minimally invasive surgery of an embodiment according to the present invention includes a positioning member 1, a looping/tightening member 2, and a reinforcing member 3. The looping/tightening member 2 can extend through the positioning member 1 to define a circled space “S” for receiving and clamping a blood vessel “V” for the purposes of temporarily blocking the flow of blood in the blood vessel “V” (see FIGS. 7 and 8).

The positioning member 1 includes a first end 1a and a second end 1b opposite to the first end 1a. The first end 1a of the positioning member 1 is connected to the looping/tightening member 2. The positioning member 1 further includes a first side and a second side opposite to the first side. The positioning member 1 further includes a through-hole 11, a positioning hole 12, and a connection gap 13 intercommunicating the through-hole 11 with the positioning hole 12. Each of the through-hole 11, the positioning hole 12, and the connection gap 13 extends from the first side through the second side of the positioning member 1. The maximum diameter of the positioning hole 12 is smaller than the maximum diameter of the through-hole 11 and is larger than the maximum diameter of the connection gap 13. The looping/tightening member 2 can extend through the through-hole 11 and can be positioned in the positioning hole 12. In this embodiment, the positioning hole 12 is located between the through-hole 11 and the first end 1a of the positioning member 1.

The positioning member 1 can further include a gripping portion 14 to be held by a hand of a surgical operator or an operation device. In this embodiment, the gripping portion 14 is located between the through-hole 11 and the second end 1b of the positioning member 1.

The positioning member 1 can be made of a biocompatible material to avoid triggering of immune responses when the positioning member 1 is placed into a human body. Preferably, the positioning member 1 is made of latex or silica gel, which is elastic to avoid destruction to the vascular clamp when pulled by an external force.

The looping/tightening member 2 includes a connection end 2a and a free end 2b. The connection end 2a is connected to the positioning member 1 by any suitable provision. For example, the connection end 2a can be connected to the first end 1a of the positioning member 1 by tight coupling, gluing, etc. Alternatively, the connection end 2a can be integrally formed with the positioning member 1 to increase the integrity of the vascular clamp, avoiding the looping/tightening member 2 from being pulled and disengaged from the positioning member 1 during a surgical procedure. In this embodiment, the looping/tightening member 2 and the positioning member 1 are made of the same biocompatible material having slight elasticity.

The looping/tightening member 2 further includes a looping section 21, a positioning section 22, and a guiding section 23. The looping section 21 is adjacent to the connection end 2a. The positioning section 22 is located between the looping section 21 and the guiding section 23. The guiding section 23 is adjacent to the free end 2b. The looping section 21 is located between the connection end 2a and the positioning section 22. The surgical operator can extend the guiding section 23 through the through-hole 11 and can position the positioning section 22 in the positioning hole 12. The looping section 21, a portion of the positioning section 22, and the positioning member 1 together define a circled space “S.” The circled space “S” is adapted for receiving the blood vessel “V.”

The positioning section 22 has a plurality of engagement blocks 221. One of the engagement blocks 221 is selectively and removably engaged in the positioning hole 12. A neck 222 is interconnected between two adjacent engagement blocks 221. Each engagement block 221 has reducing outer diameters from the connection end 2a towards the free end 2b. The maximum outer diameter of each engagement block 221 is smaller than the maximum diameter of the through-hole 11 and is larger than the maximum diameter of the positioning hole 12, such that the engagement blocks 221 can pass through the through-hole 11 and can be selectively positioned in the positioning hole 12. Furthermore, the maximum diameter of the connection gap 13 is similar to the maximum diameter of the positioning hole 12. Namely, when the maximum diameter of the connection gap 13 is slightly smaller than the maximum outer diameter of the necks 222, the necks 222 can easily pass through the connection gap 13. When the maximum diameter of the connection gap 13 is equal to or slightly larger than the maximum outer diameter of the necks 222, the necks 222 can also pass through the connection gap 13 by the elasticity of the positioning member 1 to position one of the engagement blocks 221 in the positioning hole 12.

The reinforcing member 3 is mounted between the first and second sides of the positioning member 1. Compared to the positioning member 1, the reinforcing member 3 is made of a material with toughness. The reinforcing member 3 can be mounted between the first and second sides of the positioning member 1 by injection molding or enveloping molding to increase the rigidity of the positioning member 1 for the purposes of avoiding bending during use. In this embodiment, each of the through-hole 11, the positioning hole 12, and the connection gap 13 extends through the reinforcing member 3, such that the structure of the through-hole 11, the positioning hole 12, and the connection gap 13 are less likely to deform or damage by tearing, maintaining the stability of the looping/tightening member 2 extending through and positioned by the positioning member 1.

With reference to FIGS. 7 and 8, in use of the vascular clamp in minimally invasive surgery, the surgical operator places the vascular clamp on an appropriate area of a blood vessel “V” to be clamped and extends the guiding section 23 through the through-hole 11 to wind the looping/tightening member 2 around the periphery of the blood vessel “V” to thereby form the circled space “S.” Then, the surgical operator adjusts the size of the circled space “S” according to the size of the blood vessel “V” and positions one of the engagement block 221 of the positioning section 22 in the positioning hole 12 to thereby clamp the blood vessel “V”, temporarily blocking the flow of blood in the blood vessel “V.”

If it is desired to restore the flow of the blood in the blood vessel “V” during or after the surgical procedure, the surgical operator can pass the positioning section 22 through the connection gap 13 to the through-hole 11 and then remove the positioning section 22 out of the through-hole 11. Thus, the blood vessel “V” can be loosened to permit the blood to flow again in the blood vessel “V.”

In view of the foregoing, the looping/tightening member 2 of the vascular clamp for use in minimally invasive surgery according to the present invention includes the engagement blocks 221 that can selectively be engage in the positioning hole 12 to form a circled space “S” suitable for blood vessels “V” of various diameters, reducing the time required by the surgical operator for picking suitable devices according to the diameters of the blood vessel V during the surgical procedure. Moreover, the time required by the surgical operator for loosening the blood vessel “V” can be reduced since the minimum diameter of the through-hole is larger than the maximum outer diameter of each of the plurality of engagement blocks. Thus, the time of the surgical procedure can be reduced to improve the operational efficiency and to shorten the postoperative recovery period of the patient.

Furthermore, the reinforcing member 3 mounted between the first and second sides of the positioning member 1 can avoid deformation of the through-hole 11, the positioning hole 12, and the connection gap 13 resulting from tearing, preventing disengagement of the engagement block 222 positioned in the positioning hole 12.

Thus since the invention disclosed herein may be embodied in other specific forms without departing from the spirit or general characteristics thereof, some of which forms have been indicated, the embodiments described herein are to be considered in all respects illustrative and not restrictive. The scope of the invention is to be indicated by the appended claims, rather than by the foregoing description, and all changes which come within the meaning and range of equivalency of the claims are intended to be embraced therein.

What is claimed is:

1. A vascular clamp for use in minimally invasive surgery comprising:
a positioning member including a first side and a second side opposite to the first side, with the positioning member including a through-hole, a positioning hole, and a connection gap intercommunicating the through-hole with the positioning hole, with each of the through-hole, the positioning hole, and the connection gap extending from the first side through the second side of the positioning member;
a looping/tightening member including a connection end and a free end, with the connection end connected to the positioning member, with the looping/tightening member including a positioning section having a plurality of engagement blocks, with a neck interconnected between two adjacent engagement blocks, wherein one of the plurality of engagement blocks is selectively and removably engaged in the positioning hole; and
a reinforcing member mounted between the first and second sides of the positioning member.

2. The vascular clamp for use in minimally invasive surgery as claimed in claim 1, wherein each of the through-hole, the positioning hole and the connection gap extends through the reinforcing member.

3. The vascular clamp for use in minimally invasive surgery as claimed in claim 1, with each of the plurality of engagement blocks having a maximum outer diameter smaller than a minimum diameter of the through-hole and larger than a maximum diameter of the positioning hole, and with the connection gap having a maximum diameter smaller than the maximum diameter of the positioning hole.

4. The vascular clamp for use in minimally invasive surgery as claimed in claim 1, with the positioning member further including a first end and a second end opposite to the first end, with the first end of the positioning member connected to the looping/tightening member, and with the positioning hole located between the through-hole and the first end of the positioning member.

5. The vascular clamp for use in minimally invasive surgery as claimed in claim 4, wherein the positioning member further includes a gripping portion located between the through-hole and the second end of the positioning member.

6. The vascular clamp for use in minimally invasive surgery as claimed in claim 1, with the looping/tightening member further including a looping section between the connection end and the positioning section, with the looping section, a portion of the positioning section, and the positioning member together defining a circled space, and with the circled space adapted for receiving a blood vessel.

7. The vascular clamp for use in minimally invasive surgery as claimed in claim 1, wherein each of the plurality of engagement blocks having reducing outer diameters from the connection end towards the free end.
